(19) United States
(12) Patent Application Publication
(10) Pub. No.: US 2004/0020490 A1
(43) Pub. Date: Feb. 5, 2004

(54) Device for separating gas and liquid streams, especially in tracheal catheters

(76) Inventor: Hendrik Vogt, Schwanstetten (DE)

Correspondence Address: WILLIAM COLLARD, COLLARD & ROE, P.C., 1077 NORTHERN BOULEVARD, ROSLYN, NY, 11576, US

(21) Appl. No.: 10/380,777

(22) PCT Filed: Aug. 31, 2001
(86) PCT No.: PCT/EP01/10054

(30) Foreign Application Priority Data
Sep. 19, 2000 (DE) 100 46 196.4

(51) Int. Cl. A61M 16/00; A62B 9/02

(52) U.S. Cl. 128/207.14; 128/207.16

(57) ABSTRACT

The invention relates to a device for separating gas and liquid streams, especially in tracheal catheters of patients, comprising a housing having channels for the delivery and removal of sputum. The aim of the invention is to ensure unrestricted delivery and removal of respiratory air and reliable removal of sputum. To this end, the invention provides for the arrangement of an at least spherical segment-shaped closed housing (1) comprising tubular channels (4, 5) which are located a distance apart in the area of the end surface (2) of the housing (1), each leading to a connection to the tracheal catheter and guiding through respiratory air; and a tubular channel (6) for removing sputum to the exterior, the inner front end of this channel being sealed and a suction tube (7) consisting of highly flexible or resilient elastic material lying adjacent to the inside of the channel. Said suction tube can be bent or turned autonomously by the force of gravity, extends crosswise to the sputum channel (6) and from a distance can reach approximately as far as the inner wall of the housing (1) with its free end.

The invention relates to a device for separating gas and liquid streams, especially in tracheal catheters of patients, with a housing having channels for the delivery and removal of respiratory air and the removal of sputum.

In a known device of this kind (U.S. Pat. No. specification 5,765,557), patients with tracheal catheters are provided, on the inside thereof, with a channel having paths for the delivery and removal of respiratory air, and, by means of a control device, collecting sputum can be removed via the removal channel for respiratory air, by the force of said respiratory air. The device cannot be seen from outside and the fact that its function cannot be monitored from outside is felt to be a disadvantage. It is also known (DE 198 38 370 C1) to fit the tracheal catheter with a device in which an additional opening can be controlled by a valve, via which opening the sputum can be removed by outflowing respiratory air, the valve being open for outflow of sputum during exhalation and closed during inhalation. A disadvantage of this device is that when the position of the patient changes, for example from an upright position or from the supine position, the sputum does not reach the valve-controlled opening and cannot be removed, and the sputum that cannot be removed often causes blockages of the openings for delivery and removal of respiratory air. If the patient's position changes, removal of the sputum therefore requires that the device be moved too, and this requires considerable work on the part of nursing staff and is uncomfortable and painful for the patient.

The object of the invention is to create measures which, while allowing unrestricted delivery and removal of respiratory air, also ensure reliable removal of sputum under the effect of the respiratory air and independently of the position of the patient.

According to the invention, this object is achieved by the arrangement of an at least spherical segment-shaped closed housing with tubular channels which are arranged at a distance from one another in the area of the end face of the housing and each lead to a respective connection of the channels to the tracheal catheter and are for the passage of respiratory air, and by formation of a tubular channel, for removal of sputum to the outside, whose inner front end is sealed and which has, bearing on its channel interior, a tubular suction nozzle made of a highly flexible or resiliently elastic material, which suction nozzle can be bent and/or swiveled aside automatically by the force of gravity, is designed extending transversely with respect to the sputum channel and, with its free end, reaches approximately of the inner wall of the housing. In the device, the channels serving for delivery and removal remain permanently open and free from restrictions, while, during coughing or the like, sputum passes initially into the housing, collects in the bottom zone of the sphere and from there is conveyed, by the respiratory pressure and/or expelled cough pressure, via the suction nozzle into the channel for the sputum and through the latter. The spherical shape of the housing at all times ensures, independently of the position of the patient, the formation of a bottom sphere zone acting as sump and receiving the sputum, to which sphere zone the highly flexible suction nozzle is in each case automatically connected via its free end.

The housing of the device is preferably designed as a hemisphere or as a full sphere, the channels extending in the area of the end face or in the area of the plane passing through the midpoint of the sphere. In the case of spherical segment-shaped housings, an independent attachment can be provided on the end face, which attachment receives the channels, and from which attachment the suction nozzle extends into the housing through a hole in the dividing wall between attachment and housing and can be bent or swiveled aside along its length and reaches approximately of the inner wall of the housing. The suction nozzle can be connected securely to the channel for removal of sputum or, according to an advantageous embodiment, can be designed on the channel to be freely rotatable about the longitudinal axis thereof. The suction nozzle can expediently open with its channel-side end into a pot-shaped cylinder and, by way of the cylinder, be freely rotatable on the sputum channel.

Advantageous embodiments of the device are defined by the measures in claims 7 through 22. Thus, the suction nozzle can also bear with its free end on the inner wall of the housing, in which case, in order to ensure the introduction of sputum into the suction nozzle at the housing end thereof, through-openings such as cutouts, slits or the like are necessary. Moreover, at its free end the suction nozzle can support a body of high specific weight, which body ensures automatic radial orientation of suction nozzle, or of suction nozzle and pot-shaped cylinder, with respect to the channel for sputum removal. It is also possible for the channels for removal of sputum and for passage of respiratory air to be designed to be swiveled or pivoted jointly relative to the housing.

Particularly reliable removal of sputum can be achieved using a pump which bears on the channel for removal of sputum, the inlet of the pump being connected to the suction nozzle and its outlet to the channel. Such a pump can be designed as a piston pump or a diaphragm pump whose piston or diaphragm can be brought, by respiratory pressure, from the rest position to the conveying position for sputum and, in the absence of respiratory pressure, can be returned automatically to the rest position preferably by resiliency and/or gravity and, when the inlet is closed, forces the sputum into the sputum channel. The resiliency in this case can be applied by a deformable plate body or the like made of a resiliently elastic material, or by means of rigid plate body arranged on a corrugated cuff made of resiliently elastic material, for example rubber, said plate bodies in each case being connected by force-fit to the piston or diaphragm via actuating members, for example rods.

The provision of a reflux barrier for the sputum in the sputum channel has also proven advantageous. Moreover, it is possible to achieve an increase in the cough pressure in the housing by means of a slide or flap in the channel for delivery of respiratory air, these covering the channel over part of its cross section and being automatically displaceable or pivotable, or opening in the inhalation direction, by means of outflowing respiratory air so as to free a greater cross section or the whole cross section.

Finally, the inlet end of the channel for the delivery of respiratory air can support an air filter which, if appropriate, is provided with an electrical arrangement for preheating the respiratory air and/or with moistening measures.

A preferred embodiment of the device can be obtained where the housing is formed by an outer body and an inner body, one centrically enclosing the other at a distance, the interspace between them receiving a housing-like or sponge-like filter material which, in order to form a peripheral gap by means of spacers, is delimited at a distance from the inner body by a perforated foil and, along part of its length, is provided with a recess which acts as collecting space and on which the delivery line for respiratory air bears, and it has through-openings in the outer body for introduction of incoming air into the interspace.

Finally, an alternative embodiment of the device is achieved by means of a cylindrical, closed housing which has channels for securing it on the tracheal catheter, the delivery and removal of respiratory air and the removal of sputum, the latter channel communicating with a suction nozzle which reaches of or comes close to the inner wall of the housing and receives and conveys sputum.

It will be appreciated that, by modification of the inventive concept, the device can also be used in the fields of technology and chemistry, for example for separation of air-charged mineral oils, such as waste oil from internal combustion engines.

The invention is illustrated in the figures, where:

FIG. 1 shows a device in a side view,

FIG. 2 shows a device according to FIG. 1 in a plan view,

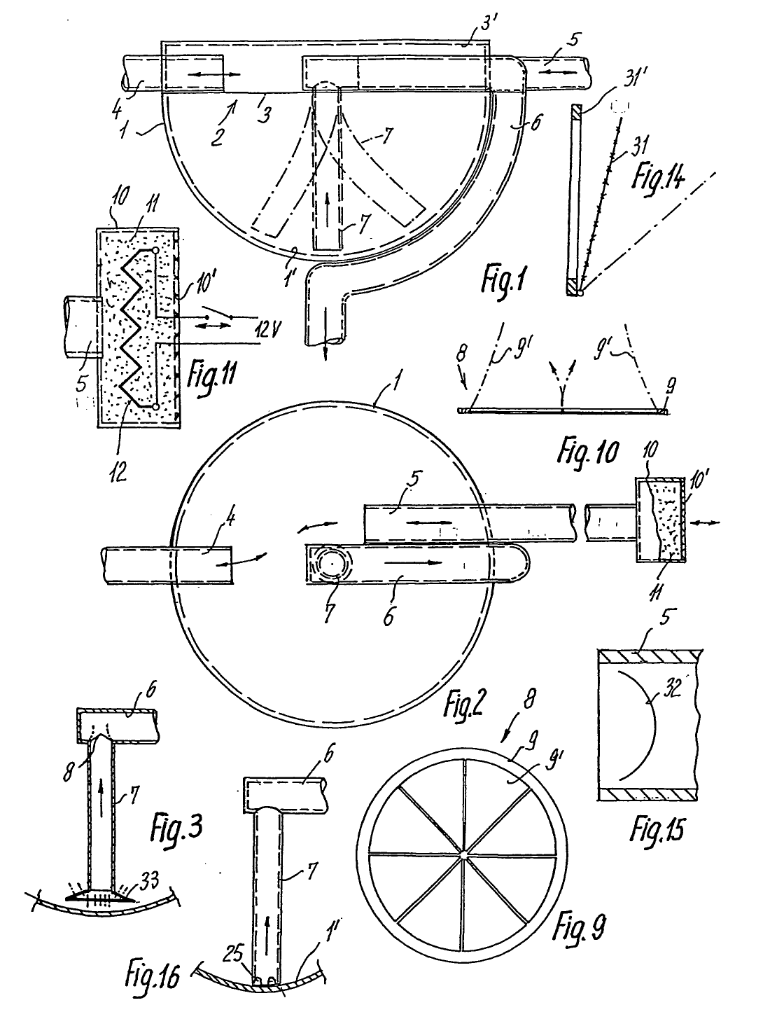

FIG. 3 shows a suction nozzle with a part of the sputum channel, in cross section,

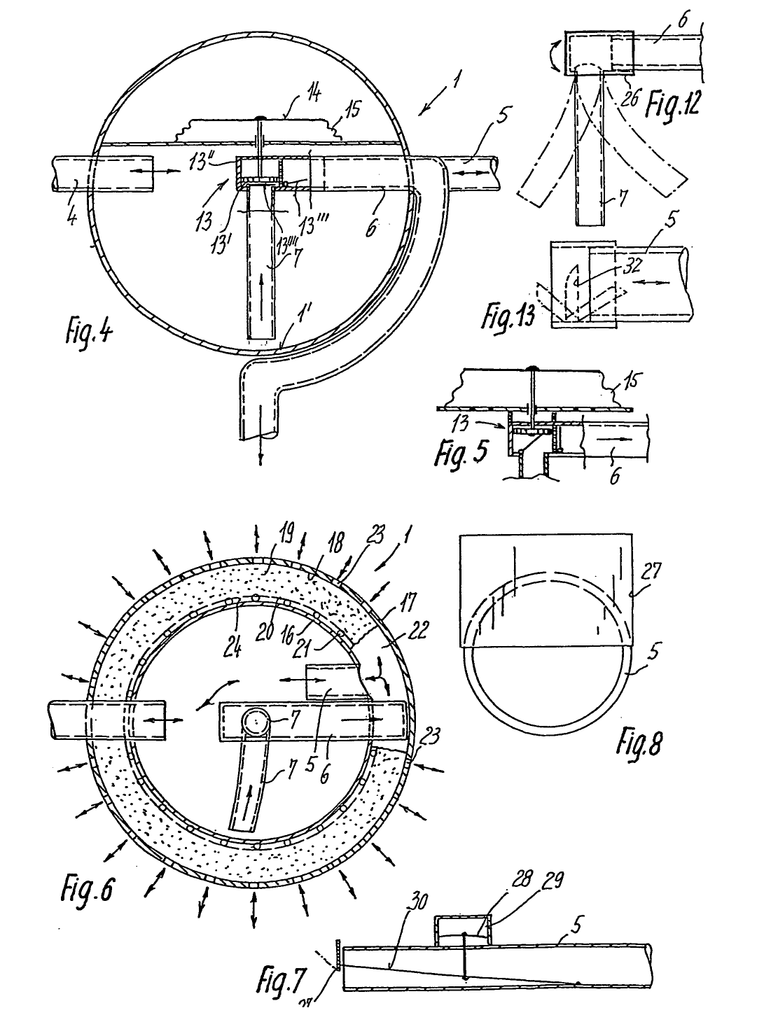

FIG. 4 shows a device with sputum pump, partially in cross section,

FIG. 5 shows a sputum pump according to FIG. 4 in another working position, in cross section,

FIG. 6 shows a device of modified design, in cross section,

FIG. 7 shows a channel for air passage, with a barrier member, in cross section,

FIG. 8 shows a barrier member in a front view, and enlarged,

FIG. 9 shows a reflux barrier in a plan view, and enlarged,

FIG. 10 shows a reflux barrier from FIG. 9 in a side view, and enlarged,

FIG. 11 shows an air filter in cross section, and enlarged,

FIG. 12 shows a suction nozzle with a part of the sputum channel in a modified design, in a side view,

FIG. 13 shows a channel portion for the passage of respiratory air with a barrier member, in a side view and enlarged,

FIG. 14 shows a barrier member according to FIG. 7 in cross section, and enlarged,

FIG. 15 shows a part of a channel portion for the passage of respiratory air with a barrier member according to FIG. 13, in cross section and enlarged, and

FIG. 16 shows a part of a suction nozzle of modified design, in a side view.

FIGS. 1 and 2 show a device with a hemispherical housing 1 which is closed by a dividing wall 3 in the area of the end face 2. The area of the end face 2 supports an attachment 3′ which receives tubular channels 4, 5 and 6. The channel 4 serves to connect the device to a tracheal catheter (not shown), while the channel 5 permits passage of respiratory air. Channel 6 is provided for the removal of sputum. Connected to the channel 6 there is a tubular suction nozzle 7 which can be bent aside and which extends transversely with respect to the central longitudinal axis of the channel 6 and is made of a highly flexible or resiliently elastic material. The suction nozzle 7 can be bent aside from the position indicated by solid lines in FIG. 1 in the plane thereof and in any desired lateral positions. When sputum enters the housing 1, for example during coughing, the sputum initially flows into the bottom sphere zone 1′ and, under the action of the respiratory air or cough pressure, passes from there into the suction nozzle 7 and onward into the channel 6 for removal to the outside.

In FIG. 3, the suction nozzle 7 is provided at its free end, on the outside, with an annular weight body 33 which ensures that the suction nozzle 7 is at all times in contact with the bottom zone 1′ of the housing 1. At its end toward the channel 6, the suction nozzle 7 has a reflux barrier 8 for sputum. In FIGS. 9 and 10, the reflux barrier 8 is formed by an annular body 9 with a number of segments 9′ which can swing open and which, under the effect of respiratory pressure, are bent upward by sputum and, at the end of the respiratory pressure acting on the sputum, are closed by means of the reflux barrier 8 dropping back. The channel 5 has an air filter 10 on the outside, for example with a filter body 11 of foam or the like, which air filter, in the embodiment according to FIG. 11, is closed in the area of the open face by a grid 10′ and is further equipped with a heating arrangement. The heating arrangement is formed simply by a resistance wire 12 which can preferably be connected to a low-voltage source.

FIG. 4 shows a device with a spherical housing 1 which once again has channels 4 through 6 and in which the sputum channel 6 is additionally equipped with a pump 13. In this illustrative embodiment, the pump 13 is designed as a piston pump which sucks the sputum through the suction nozzle 7 and conveys it into the channel 6. The piston 13′ of the pump 13 is connected to a plate body 14 which engages on a corrugated cuff 15 secured to the housing and made of resiliently elastic material.

In the event of coughing, sputum passes from the bottom spherical zone 1′ of the housing 1 into the suction nozzle 7, through the inlet valve 13″″ into the pump cylinder 13″, and through the outlet valve 13′″ into the channel 6. In this process, the plate body 14 is initially moved upward (FIG. 5) together with the piston 13′ counter to the prestressing of the cuff 15 and, when the coughing or the like ceases, is moved back down to the rest position by the prestressing of the cuff 15.

In the illustrative embodiment in FIG. 6, as a modification, the housing 1 of the device is formed by two spherical bodies 16, 17, one enclosing the other at a distance, and the interspace 18 between the two spherical bodies 16, 17 receives a filter material 19 delimited on the inside by a perforated foil 20. The foil 20 is supported by spacers 21 so as to form a gap 24 from the inner spherical body 16. The filter material 19 is provided with a recess 22 on which the channel 5 for passage of respiratory air bears. Whereas the inner spherical body 16 in the illustrative embodiment is closed, the outer spherical body 17 is provided with air delivery openings 23. The respiratory air can thus pass through the filter material 19 via the air delivery openings 23 and pass into the gap 24 and onward into the recess 22. In this embodiment too, the channel 6 for removal of sputum is provided with a suction nozzle 7 via which the sputum passes from the inner spherical body 16 into the channel 6.

It is conceivable to design the suction nozzle 7 with its free end bearing on the inner wall of the housing 1, in which case, for introducing sputum into the suction nozzle 7, through-openings 25 or cutouts are provided at the free end thereof (FIG. 16).

In FIG. 12, a pot-shaped sleeve 26 is mounted rotatably on the sputum channel 6, and the channel-side end of the suction nozzle 7 opens into this sleeve. By means of said sleeve 26, the suction nozzle 7 can be swiveled through 360° on the sputum channel 6 and additionally, because of its flexibility, can be bent out to both sides from the straight position. In this way it is possible to ensure that the whole inner wall of the respective housing 1, 16, 17 can be contacted and the removal of sputum guaranteed.

FIGS. 7 and 8 show a channel 5 for the passage of respiratory air, which channel is blocked, over part of its cross section, by a slide 27 or flap. By means of the barrier slide 27, it is possible, in the event of coughing, to obtain a pressure increase in the housing 1, which pressure increase ensures reliable introduction of sputum into the suction nozzle 7 and transporting of the sputum into the channel 6. The slide 27 acting as barrier can be displaced by respiratory air so as to free the channel 5, for which purpose the respiratory air strikes a membrane 28 in the cylinder 29 and lifts the slide 27 via a rod 30. A flat closed or curved plate (FIG. 8) or a screen body 31 with frame 31′ can also serve as barrier.

In FIGS. 13 and 14, a tongue 32 acting as a baffle and made of a thin-walled flexible material is fixed via one end in the channel 5 and, under the effect of respiratory air, can be tilted in one or other direction of movement. The baffle 32 acts as a barrier member and is arc-shaped in cross section, as a result of which the baffle can be tilted in one direction of movement of the respiratory air with less pressure than in the other direction of movement of the respiratory air. In the absence of respiratory air flow, the baffle 32, under the effect of material stresses, sets itself automatically to the upright position shown in FIG. 13.

1. A device for separating gas and liquid streams, especially in tracheal catheters of patients, with a housing having channels for the delivery and removal of respiratory air and for the removal of sputum, characterized by the arrangement of an at least spherical segment-shaped closed housing (1) with tubular channels (4, 5) which are arranged at a distance from one another in the area of the end face (2) of the housing (1) and each lead to a respective connection of the channels to the tracheal catheter and are for the passage of respiratory air, and of a tubular channel (6), for removal of sputum to the outside, whose inner front end is sealed and which has, bearing on its channel interior, a tubular suction nozzle (7) made of a highly flexible or resiliently elastic material, which suction nozzle (7) can be bent and/or swiveled aside automatically by the force of gravity, is designed extending transversely with respect to the sputum channel (6) and, with its free end, reaches approximately of the inner wall of the housing (1).

2. The device as claimed in claim 1, characterized in that the housing (1) is designed as a hemisphere and the channels (4, 5, 6) are arranged in the area of the end face (2) of the housing (1).

3. The device as claimed in claim 1, characterized in that the housing (1) is designed as a full sphere and the channels (4, 5, 6) are arranged in a plane passing through the area of the midpoint of the sphere.

4. The device as claimed in claim 1, characterized in that the housing (1) is spherical segment-shaped, in particular hemispherical, and has, separately and independently, on the end face, a chamber-like attachment (3′) which receives the connection channel (4) to the tracheal catheter and the channel 5 for passage of respiratory air and the channel (6) for removal of sputum, and the suction nozzle (7) extends into the housing (1) through a hole in the dividing wall (3) between attachment (3′) and housing (1), and, being able to be bent or swiveled aside along its length, is guided approximately of the inner wall of the housing (1).

5. The device as claimed in claim 1, characterized in that the suction nozzle (7) is designed to be freely bendable and, with the channel-side end arranged tightly on the channel (6) for removal of sputum, is designed to be freely rotatable about the longitudinal axis of the channel (6).

6. The device as claimed in claim 5, characterized in that the suction nozzle (7) opens with its channel-side end into a pot-shaped sleeve (26) and, by way of the sleeve, is freely rotatable on the sputum channel (6) about the central longitudinal axis thereof.

7. The device as claimed in claim 1, characterized in that the channels (6, 5) for the removal of sputum and the passage of respiratory air are designed to be jointly pivotable or rotatable relative to the housing (1).

8. The device as claimed in claims 1, 2, 5 and 6, characterized in that the suction nozzle (7) bears with its free end on the inner wall of the housing (1) and, at the end facing the inner wall, has through-openings (25) or the like for introduction of sputum into the suction nozzle (7).

9. The device as claimed in claims 5 and 6, characterized in that, on the outside of the free end, the suction nozzle (7) supports a body, for example an annular body (33), of high specific weight, and in that, by means of this weight body (33), the suction nozzle (7), or the suction nozzle (7) jointly with sleeve (26), can be brought to and held in the housing (1) at a radial orientation with respect to the channel (6) for sputum removal.

10. The device as claimed in claim 1, characterized in that a pump (13) bears with its outlet (13′″) on the channel (6) for removal of sputum, its inlet (13″″) being connected to the suction nozzle (7).

11. The device as claimed in claim 10, characterized in that the pump (13) is designed as piston pump or diaphragm pump, and in that the piston (13′) or the diaphragm of the pump (13) can be brought, by respiratory pressure, from the rest position to the conveying position for sputum and, in the absence of respiratory pressure, can be returned automatically to the rest position by resiliency and/or gravity.

12. The device as claimed in claim 11, characterized in that the resiliency can be formed by a deformable plate body or the like made of a resiliently elastic material, which plate body is fixed on the housing (1) or the channel (6) for of sputum and is securely connected to the piston or the diaphragm of the pump by an actuating member.

13. The device as claimed in claim 11, characterized in that the resiliency can be applied by a rigid plate body (14) or the like arranged on a corrugated cuff (15) made of resiliently elastic material, and the plate body (14) is connected to the piston (13′) or the diaphragm of the pump (13) by an actuating member.

14. The device as claimed in claim 1, characterized in that the channel (6) for removal of sputum has, in order to avoid inadvertent reflux of sputum, a reflux barrier, for example a lamellar shutter or the like, with a barrier action oriented in the reflux direction (8).

15. The device as claimed in claim 1, characterized in that, in the rest position, the channel (5) for delivery of respiratory air is covered over part of its cross section by a slide (27) or a flap, and in that the slide or the flap can be automatically displaced or pivoted, or opened in the inhalation direction, by outflowing respiratory air and/or coughs so as to free a greater cross section or the whole cross section of the channel (5) for respiratory air.

16. The device as claimed in claim 1, characterized in that, in the rest position, the channel (5) for delivery of respiratory air is covered over part of its cross section by a tongue (32) made of flexible or resiliently elastic material, the tongue (32) can be tilted by incoming respiratory air so as to increase the cross section of the passage of respiratory air and can be swiveled back by outflowing respiratory air counter to the deformation resistance.

17. The device as claimed in claim 1, 14, 15, 16, characterized in that the inlet end of the tubular channel (6) for the passage of respiratory air has an air filter (10) on the outside.

18. The device as claimed in claims 1 and 17, characterized in that the air filter (10), for the purpose of preheating incoming respiratory air, has an electrical heating arrangement, for example a heating coil (12) or the like, which can either be switched on manually at periods or can be switched on automatically via a sensor.

19. The device as claimed in claims 17 and 18, characterized in that the filter body of the air filter (10) and/or the incoming respiratory air can be moistened with liquid media via openings in the filter housing or semipermeable wall portions in the sputum channel (6).

20. The device as claimed in claims 1 through 3, characterized in that a liquid medium acting as flushing agent can be introduced into the housing interior, for example by a pump or the like.

21. The device as claimed in claim 1, characterized in that the housing (1) is formed by an outer body (17) and an inner body (16), for example spherical bodies, one centrically enclosing the other at a distance, in that the interspace (18) between the two bodies (16, 17) receives a foam-like or sponge-like filter material (19) which is delimited at a distance from the inner body (16) by a foil (20), said foil (20) being supported by spacers (21) on the inner body (16) in order to form a peripheral gap (24) and being provided with a number of air through-openings, the filter material (19) being left open along part of its length, and in that the channel (5) for the passage of respiratory air bears on the recess (22), and the outer body (17) is provided with through-flow openings (23) for the respiratory air.

22. The device as claimed in claims 1 and 5 through 14, characterized in that the housing is formed by a cylinder closed at the end faces and on the circumferential surfaces, the housing has channels formed in the end faces for securing it on the tracheal catheter, of the passage of respiratory air and removal of sputum to the outside, the channel for removal of sputum is sealed at the inner end and communicates with an automatically bendable and/or pivotable tubular suction nozzle made of a highly flexible or resiliently elastic material, which is designed extending transversely with respect to the tubular sputum channel and with its free end approximately reaches as far as or bears on the inner wall of the housing.
